(19) United States
(12) Patent Application Publication
(10) Pub. No.: US 2008/0095711 A1
(43) Pub. Date: Apr. 24, 2008

(54) Modulators of Pulmonary Hypertension

(76) Inventors: John R. Falck, Dallas, TX (US); Alan H. Stephenson, St. Louis, MO (US)

Correspondence Address: THOMPSON COBURN, LLP, ONE US BANK PLAZA, SUITE 3500, ST LOUIS, MO, 63101, US

(21) Appl. No.: 11/848,396

(22) Filed: Aug. 31, 2007

Related U.S. Application Data
(60) Provisional application No. 60/824,201, filed on Aug. 31, 2006.

(51) Int. Cl. A61K 31/201; A61K 31/192; A61P 9/12; C07C 59/58; A61K 31/197

(52) U.S. Cl. 424/9.2; 514/559; 514/560; 514/563; 514/571; 554/219

(57) ABSTRACT

Compounds, compositions, and methods for inhibiting pulmonary hypertension are disclosed. The invention is particularly directed to the use of agents that specifically inhibit the activity of certain endogenously produced epoxyeicosatrienoic acids that promote vasoconstriction of pulmonary arteries. These agents are particularly useful for inhibiting hypoxia-induced pulmonary hypertension. The invention further discloses additional compounds, compositions and methods for increasing pulmonary hypertension.

### CROSS-REFERENCE TO RELATED APPLICATIONS

This application claims priority to U.S. Provisional Patent Application Ser. No. 60/824,201, filed Aug. 31, 2006, which is incorporated by reference herein in its entirety.

### STATEMENT REGARDING FEDERALLY SPONSORED RESEARCH OR DEVELOPMENT

National Institutes of Health grant GM31278 to J. R. Falck.

### APPENDIX

Not Applicable

### BACKGROUND OF THE INVENTION

1. Field of the Invention

This invention relates generally to compositions and methods for inhibiting pulmonary hypertension and, more particularly, to methods of inhibiting epoxyeicosatrienoic acid (EET)-mediated vasoconstriction of pulmonary arteries. The methods described by the invention are useful in relieving subjects of the undesirable symptoms and life threatening outcomes of pulmonary hypertension. The methods are also useful for evaluating and optimizing new treatments for controlling pulmonary hypertension. This invention further relates to methods of increasing pulmonary hypertension.

2. Related Art

Pulmonary hypertension is a life threatening condition that occurs when the arteries that supply oxygenated blood to the lungs become constricted. Although the causes of pulmonary hypertension are varied, the consequences of this condition are consistent. Early symptoms of pulmonary hypertension include shortness of breath, lack of stamina, fainting, and chest pain. Left untreated, pulmonary hypertension typically results in heart failure and death.

At present, the most common treatment for pulmonary hypertension is continuous intravenous administration of prostacyclin. Although significant reductions in pulmonary hypertension are observed with this method, continuous administration of this drug by pumps is inconvenient and also results in a variety of side effects such as headache, jaw pain, leg pain, and diarrhea (Barst R. J., et al. Ann Intern Med 1994; 121:409-415). To circumvent the side effects associated with intravenous administration, prostacyclin and more stable prostacyclin analogues such as iloprost have also been delivered directly to the lungs by inhalation (Olschewski, H. et al., N Engl J Med. 2002 Aug. 1; 347(5):322-329). Although promising results have been obtained with the more stable prostacyclin analogue iloprost, long term iloprost therapy is inconvenient in that it entails 6-9 inhalation sessions per day with each session lasting up to about 12 minutes (when a conventional jet nebulizer is used) or 4 minutes (when an ultrasonic nebulizer is used; Gessler T., et al., Eur Resp J 2001; 17:14-19). Given these drawbacks, alternative therapies for pulmonary hypertension are clearly called for.

Inhibitors of the endogenously produced agents that cause pulmonary hypertension represent a potentially novel approach to the treatment of this disease. In the lung, four epoxyeicosatrienoic acid (EET) regioisomers (5,6-, 8,9-, 11,12-, and 14,15-EET) typically constrict the small diameter intralobar pulmonary arteries (PA) and can thus cause pulmonary hypertension. In contrast, the EET regioisomers dilate systemic blood vessels located outside of the lung. It is believed that EETs may induce pulmonary hypertension under certain conditions. The following findings are consistent with a role for 5,6-EET in hypoxia induced hypertension: 1) 5,6-EET increased pulmonary vessel resistance in isolated perfused lungs (Stephenson, A. H. et al., American Journal of Physiology 284: H2153-H2161, 2003); 2) 5,6-EET was reported to contract isolated pressurized intrapulmonary arterial vessels of rats and rabbits (Zhu, et al. American Journal of Physiology—Lung Cellular & Molecular Physiology 278: L335-L343, 2000, Yaghi, et al. Journal of Pharmacology & Experimental Therapeutics 297: 479-488, 2001). Although it has been reported that 5,6-EET may under certain conditions exhibit vasodilatory effects in canine pulmonary arteries (Stephenson, A. H., et al., American Journal of Physiology 275:H100, 1998), in general, EETs typically exert a vasoconstrictive effect in pulmonary arteries; 3) selective inhibition of epoxygenase activity with N-methyl sulfonyl-6-(2-propargyloxyphenyl)hexanamide (MSPPOH) in mice, significantly reduced acute hypoxia-induced pulmonary hypertension and chronic hypoxia-induced pulmonary vascular remodeling (Pokreisz et al., Hypertension 27:762-770, 2006). Given this evidence, agents that specifically inhibit the action of endogenously produced EETs emerge as novel candidates for the control of pulmonary hypertension in subjects other than canines.

### SUMMARY OF THE INVENTION

It is in view of the above problems that the present invention was developed. The invention is first related to a series of novel compounds that are useful in treating pulmonary hypertension and/or inhibiting 5,6-EET-mediated vasoconstriction of pulmonary arteries. This class of compounds is represented by the following generic formula: 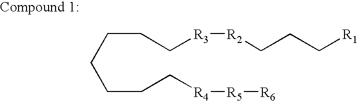
wherein R₁ is selected from the group consisting of consisting of —COOH, —C(O)O—(C₁-C₅ alkyl), —C(O)O—(C₁-C₅ alkenyls), —C(O)O—(C₁-C₅ alkynyl), —C(O)O—(C₃ cyclopropyl), —C(O)NHSO₂CH₃, —C(O)C₁-C₅ alkyl, —C(O)C₁-C₅ alkenyls, —C(O)C₁-C₅ alkynyl, —C(O)C₃ cyclopropyl, —C(O)NHSO₂CH₃, —C(O)NHCH₂COOH, —C(O)NHCHCH₃COOH, —C(O)NHC(CH(CH₃)₂)COOH, —C(O)NHC(CH₂OH)COOH —NHC(CH(CH₃)OH)COOH, —C(O)OCHCHOHCH₂OH), a tetrazole, a 5-substituted-2,4-thiazolidinedione, an oxo-oxadiazole, an oxo-thiadiazole, a mercaptoazole, a sulfinylazole, a sulfonylazole, an isoxazole, an isothiazole, a hydroxy-thiadiazole, a hydroxy-chromone, a phosphinate, a phosphonate, a phosphonamide, a sulphonate, a sulphonamide, an acyl-sulphonamides and —C(O)O—(CH₂—CH₂—O)ₙ—H, where n is between 1 and 15,
wherein R2 and R3 are —CHₓ—, x is either zero or one, and wherein R2 and R3 linked by a double or a triple bond;
wherein R4 is —O— when R5 is —CH₂— and R6 is an unbranched C₁-C₅ alkyl;
wherein R4 is —CH₂— when R5 is —O— and R6 is an unbranched C₁-C₅ alkyl;
and wherein R4 and R5 are —CH₂— when R6 is an unbranched —O—C₁-C₅ alkyl.

Pharmaceutically acceptable salts, esters, and acid derivatives of the foregoing compounds are further contemplated by the instant invention. In this regard, when R₁ is —COOH (carboxylic acid), pharmaceutically acceptable salts, esters, amides and acid derivatives of that carboxylic acid group are anticipated. Salts of that R₁ group would include ammonium salts, alkali metal salts such as sodium and potassium salts, alkaline earth metal salts such as calcium and magnesium salts, organic base salts such as dicyclohexylamine and N-methyl-D-glucamine, and salts with amino acids such as arginine and lysine. Esters of that R₁ group would include lower alkyl esters such as —C₁-C₅ alkyl esters, lower alkenyl esters such as —C₁-C₅ alkynyl esters, lower alkynyl esters such as C₁-C₅ alkenyl esters, hydroxy (lower) alkyl ester, lower alkoxy (lower) alkyl esters, aryl esters, aryl (lower) alkyl esters, glycerol esters and polyethylene glycol esters. Acid derivatives of that R₁ group would include —C₁-C₅ alkyl amide, C₁-C₅ alkynyl amides, C₁-C₅ alkenyl amides, amino acid amides, hydroxamic acid (—NHOH), acyl-cyanamide (—NHCN), acylsulfonamides (—CO—NH—SO₂—R′), and methylsulfonimides. Synthesis of the ester and acid derivatives described herein can be performed by methods described in Carey, F. A. and Sundberg, R. J. Advanced Organic Chemistry, Fourth Edition—Part B: Reaction and Synthesis, 2001.

Such compounds described by Compound 1 and the preceding description are hereinafter referred to as “compounds of the invention”.

At least one exemplary compound of the invention, 13-heptyloxytridec-5(Z)-enoic acid or 13-HTEC, that is useful in treating pulmonary hypertension and/or inhibiting 5,6-EET-mediated vasoconstriction of pulmonary arteries is shown below. This compound is represented by the structure following immediately below:

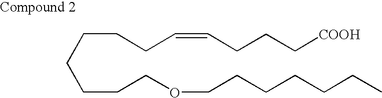

Pharmaceutically acceptable salts, esters, and acid derivatives of the 13-HTEC are further contemplated by the instant invention. In particular, —C₁-C₅ alkyl ester, —C₁-C₅ alkenyl ester, —C₁-C₅ alkynyl ester, —CHCHOHCH₂OH ester, glycerol ester, polyethylene glycol ester, —NHSO₂CH₃ amide, —NHCH₂COOH amide, —NHCHCH₃COOH amide, —NHC(CH(CH₃)₂)COOH amide, —NHC(CH₂OH)COOH amide, —NHC(CH(CH₃)OH)COOH amide, —C₁-C₅ alkyl amide, —C₁-C₅ alkynyl amides, C₁-C₅ alkenyl amides, amino acid amides, hydroxamic acid (—NHOH), acyl-cyanamide (—NHCN), acylsulfonamide (—CO—NH—SO₂—R′), and methylsulfonimide derivatives of the free carboxylic acid group of 13-HTEC are also contemplated as being useful in the practice of the instant invention. Polyethylene glycol ester derivatives of the free carboxylic acid group of 13-HTEC are represented herein by the formula —O—(CH₂—CH₂—O)ₙ—H, where n is between 1 and 15.

Another exemplary compound of the invention, 14-(hexyloxy)tetradec-5-ynoic acid or 14-HTYC has also been shown to be useful in treating pulmonary hypertension and/or inhibiting 5,6-EET-mediated vasoconstriction of pulmonary arteries. This compound is represented by the structure:

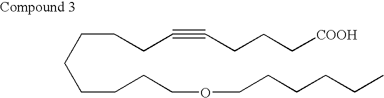

Pharmaceutically acceptable salts, esters, amides and acid derivatives of the 14-HTYC are further contemplated by the instant invention. In particular, —C₁-C₅ alkyl ester, —C₁-C₅ alkenyl ester, —C₁-C₅ alkynyl ester, —CHCHOHCH₂OH ester, glycerol ester, polyethylene glycol ester, —NHSO₂CH₃ amide, —NHCH₂COOH amide, —NHCHCH₃COOH amide, —NHC(CH(CH₃)₂)COOH amide, —NHC(CH₂OH)COOH amide, —NHC(CH(CH₃)OH)COOH amide, —C₁-C₅ alkyl amide, —C₁-C₅ alkynyl amides, C₁-C₅ alkenyl amides, amino acid amides, hydroxamic acid (—NHOH), acyl-cyanamide (—NHCN), acylsulfonamide (—CO—NH—SO₂—R′), and methylsulfonimide derivatives of the free carboxylic acid group of 14-HTYC are also contemplated as being useful in the practice of the instant invention. Polyethylene glycol ester derivatives of the free carboxylic acid group of 14-HTYC are represented herein by the formula —O—(CH₂—CH₂—O)ₙ—H, where n is between 1 and 15.

The present invention further relates to the use of Compound 1, 13-HTEC, 14-HTYC, and pharmaceutically acceptable salts, esters, amides or acid derivatives thereof, in the manufacture of a medicament for the treatment of pulmonary hypertension.

Compositions suitable for administering the compounds of the invention either orally, topically, rectally, percutaneously, by parenteral injection, intranasally or by inhalation are further contemplated for use in treating pulmonary hypertension or in the manufacture of a medicament for the treatment thereof. Such compositions are comprised of one or more compounds of the invention, pharmaceutically acceptable salts, esters or acid derivatives thereof, and pharmaceutically acceptable carriers. Pharmaceutically acceptable carriers specifically adapted for administering compositions containing one or more compounds of the invention by inhalation are particularly envisioned.

Methods of treating pulmonary hypertension are also contemplated. Such methods comprise the step of administering to a subject suffering from pulmonary hypertension a composition comprising a pharmaceutically acceptable carrier and at least one compound of the invention, pharmaceutically acceptable salts thereof, pharmaceutically acceptable esters thereof, pharmaceutically acceptable acid derivatives thereof, and a pharmaceutically acceptable carrier, thereby decreasing pulmonary hypertension in the treated subject. In certain instances, the compound of the invention may be 13-heptyloxytridec-5(Z)-enoic acid (13-HTEC), 14-(hexyloxyl)tetradec-5-ynoic acid as well as pharmaceutically acceptable salts, esters, acid derivatives and combinations thereof. Pulmonary hypertension brought on by any number of factors including but not limited to congenital heart disease, collagen vascular tissue disease such as scleroderma, lupus, rheumatoid arthritis, infections, use of diet drugs or use of illegal drugs may be treated by this method. Treatment of pulmonary hypertension caused by hypoxia, including cases where hypoxia is induced by exposure to high altitudes, is also envisioned. The subject treated for pulmonary hypertension may be a mammal selected from the group consisting of a rabbit, a pig, a rat, a horse, a cow or a human. In these methods, the composition containing one or more compounds of the invention may be administered to the subject orally, topically, rectally, percutaneously, by parenteral injection, intranasally or by inhalation. Practice of this invention by administering the compound by inhalation with a nebulizer or inhaler is specifically contemplated.

Methods of inhibiting 5,6-epoxyeicosatrienoic acid (EET)-mediated vasoconstriction of pulmonary arteries are also contemplated by this invention. These methods comprise the step of administering a composition comprising an acceptable carrier and at least one compound of the invention; thereby inhibiting 5,6-epoxyeicosatrienoic acid (EET)-mediated vasoconstriction of pulmonary arteries. In certain instances, the compound of the invention may be 13-heptyloxytridec-5(Z)-enoic acid (13-HTEC), 14-hexyloxyltetradec-5-ynoic acid, as well as pharmaceutically acceptable salts, esters, amides, acid derivatives and combinations thereof. Pulmonary vasoconstriction caused by production in the 5,6-epoxyeicosatrienoic acid (EET) in a subject is inhibited by administering to the subject the compounds of the invention either orally, topically, rectally, percutaneously, by parenteral injection, intranasally or by inhalation. Practice of this invention by administering the compound of the invention by inhalation with a nebulizer, inhaler, dry powder inhaler, or metered dose inhaler is specifically contemplated. The subject treated for pulmonary hypertension may be a mammal selected from the group consisting of a rabbit, a pig, a rat, a horse, a cow or a human. Pulmonary vasoconstriction caused by exposure of pulmonary arteries to 5,6-epoxyeicosatrienoic acid (EET) to the compounds of the invention and a suitable carrier in an ex vivo system is also contemplated. The ex vivo system may be an intralobar pulmonary artery system derived from a rabbit, rat, or pig. Alternatively, the ex vivo system may be an isolated perfused lung system derived from a rabbit, rat, or pig.

Finally, methods of increasing pulmonary vasoconstriction are also contemplated by the invention. Methods of increasing pulmonary vasoconstriction are indicated in certain pathological conditions wherein a subject is in need of increased pulmonary vasoconstriction to reverse the loss of normal pulmonary vascular reactivity and/or to restore optimal ventilation-perfusion relationships. Inflammation, pneumonia, sepsis, and Adult Respiratory Distress Syndrome (ARDS) are non-limiting examples of pathological conditions wherein a subject is in need of increased pulmonary vasoconstriction. To increase pulmonary vasoconstriction in a subject in need thereof, a composition comprising the compound 14,15-epoxyeicosa-5(Z)-enoic acid (14,15-EEZE), pharmaceutically acceptable salts thereof, pharmaceutically acceptable esters thereof, pharmaceutically acceptable acid derivatives thereof and a pharmaceutically acceptable carrier is administered, thereby increasing pulmonary vasoconstriction in said subject. The structure of 14,15-EEZE is shown below:

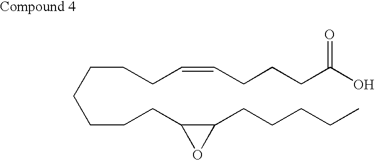

Further features and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 illustrates a series of EET analog compounds and summarizes their 5,6-EET antagonist activity.

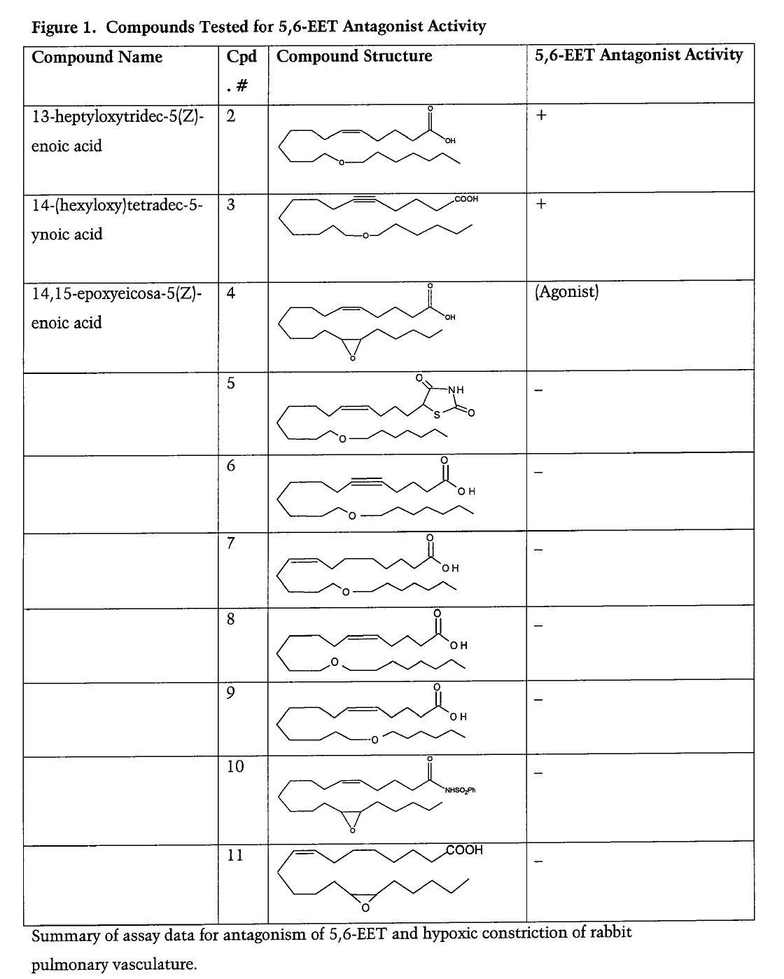

FIG. 2 illustrates a continuation of the series of EET analog compounds of FIG. 1 and summarizes their 5,6-EET antagonist activity.

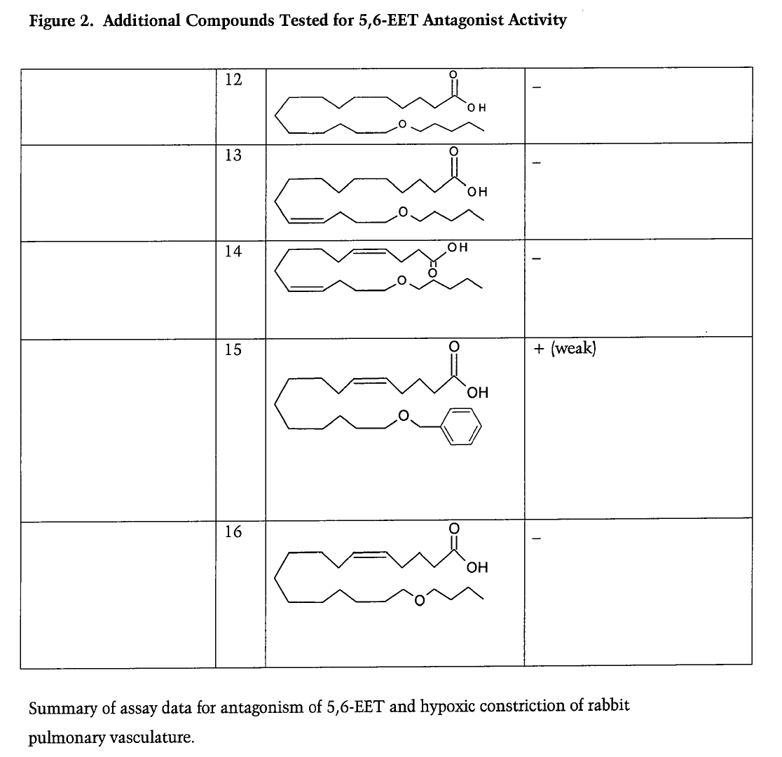

FIG. 3 illustrates the ability of 13-HTEC (10 μM) to specifically inhibit 5,6-EET-induced contraction of rabbit pulmonary arteries (leftmost panel). However, the inhibitory effect of 13-HTEC is specific in that 14,15-EET, PGF_{2α}- or 5-hydroxytryptamine (5-HT) induced pulmonary vasoconstriction is not inhibited by 13-HTEC at 10 μM. Minimal effects of 13-HTEC on norepinephrine induced vasoconstriction are observed.

FIG. 4 illustrates the ability of 13-HTEC to inhibit hypoxia-induced pulmonary vasoconstriction in isolated perfused rabbit lungs. When the isolated perfused rabbit lungs are treated with the nitric oxide synthesis inhibitor L-NAME in the absence of oxygen, hypoxia induced vasoconstriction is observed (see condition 2 in sections A and B). However, when 13-HTEC at 10 μM is provided, hypoxia-induced vasoconstriction is reduced.

FIG. 5 illustrates the pulmonary vasoconstrictive effect of the compound 14,15-EEZE in the presence of the pulmonary vasodilator 5,6-EET. The increase in Active Tension (g) is plotted against increased concentrations of 5,6-EET in the presence of either 14,15-EEZE (1 μM) or vehicle.

FIG. 6 illustrates a series of proposed compounds that can be used to inhibit 5,6-EET-mediated pulmonary hypertension.

### DETAILED DESCRIPTION OF THE PREFERRED EMBODIMENTS

### Definitions

“Acceptable carrier” refers to a carrier that is not deleterious to the other ingredients of the composition and is not deleterious to the subject to which it is administered.

“Pharmaceutically acceptable carrier” refers to a carrier that is not deleterious to the other ingredients of the composition and is not deleterious to the human or other animal recipient thereof. In the context of the other ingredients of the composition, “not deleterious” means that the carrier will not react with or degrade the other ingredients or otherwise interfere with their efficacy. Interference with the efficacy of an ingredient does not encompass mere dilution of the ingredient. In the context of the animal host or subject, “not deleterious” means that the carrier is not injurious or lethal to the animal.

“Acid derivative” is used herein to describe —C₁-C₅ alkyl amide, C₁-C₅ alkynyl amide, C₁-C₅ alkenyl amide, amino acid amide, hydroxamic acid (—NHOH), acyl-cyanamide (—NHCN), acylsulfonamide (—CO—NH—SO₂—R′), and methylsulfonimide derivatives of either the R₁ group of Compound 1 when R₁ is —COOH or the C₁ carboxylic acid group of compounds such as 13-HTEC or 14-HTYC.

“Administration” refers to any means of providing a compound or composition to a subject. Non-limiting examples of administration means include oral, topical, rectal, percutaneous, parenteral injection, intranasal and inhalation delivery.

“Inhalation delivery” refers to the administration of an aerosolized composition containing the active compound or compounds of the invention to a subject that draws the aerosolized composition through their nose and/or mouth by breathing.

“Therapeutically effective amount” refers to the amount of compound or composition that will yield the desired effect on the condition intended to be treated or prevented. In the case of a subject being treated for pulmonary hypertension, a therapeutically effective amount is that amount that will reduce pulmonary hypertension to any level that is beneficial to the subject. Ideally, pulmonary hypertension is reduced to levels that are in the range of values considered normal for a healthy subject residing at the given altitude.

“Pulmonary hypertension” refers to a condition where a subject's pulmonary arterial blood pressures are elevated relative to the normal range of values for healthy individuals of similar age, sex and weight who live at the same altitude and are engaged in similar activities. Pulmonary hypertension is typically assessed by measuring pulmonary arterial pressure (Ppa), but may also be assessed by determining left atrial pressure (PLA), central venous pressure (PCV), systemic arterial pressure (PSYS), heart rate (HR), cardiac output (CO), pulmonary artery wedge pressure (Ppa,we), right ventricular ejection fraction (RVEF), and central venous oxygen saturation (Sv_{O2}).

“Subject in need thereof” refers to mammal that would benefit from reductions in pulmonary hypertension.

“Vasoconstriction” refers to any decrease in the diameter of a vessel.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

Compounds Used in the Invention

One embodiment of the instant invention is a series of novel compounds that are useful in treating pulmonary hypertension and/or inhibiting 5,6-EET-mediated vasoconstriction of pulmonary arteries. This class of compounds is represented by the following generic formula: 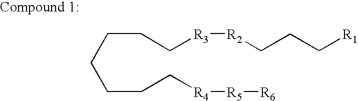
wherein R₁ is selected from the group consisting of consisting of —COOH, —C(O)O—(C₁-C₅ alkyl), —C(O)O—(C₁-C₅ alkenyls), —C(O)O—(C₁-C₅ alkynyl), —C(O)O—(C₃ cyclopropyl), —C(O)NHSO₂CH₃, —C(O)C₁-C₅ alkyl, —C(O)C₁-C₅ alkenyls, —C(O)C₁-C₅ alkynyl, —C(O)C₃ cyclopropyl, —C(O)NHSO₂CH₃, —C(O)NHCH₂COOH, —C(O)NHCHCH₃COOH, —C(O)NHC(CH(CH₃)₂)COOH, —C(O)NHC(CH₂OH)COOH —NHC(CH(CH₃)OH)COOH, —C(O)OCHCHOHCH₂OH), a tetrazole, a 5-substituted-2,4-thiazolidinedione, an oxo-oxadiazole, an oxo-thiadiazole, a mercaptoazole, a sulfinylazole, a sulfonylazole, an isoxazole, an isothiazole, a hydroxy-thiadiazole, a hydroxy-chromone, a phosphinate, a phosphonate, a phosphonamide, a sulphonate, a sulphonamide, an acyl-sulphonamides and —C(O)O—(CH₂—CH₂—O)ₙ—H, where n is between 1 and 15,
wherein R2 and R3 are —CHₓ—, x is either zero or one, and wherein R2 and R3 linked by a double or a triple bond;
wherein R4 is —O— when R5 is —CH₂— and R6 is an unbranched C₁-C₅ alkyl;
wherein R4 is —CH₂— when R5 is —O— and R6 is an unbranched C₁-C₅ alkyl;
and wherein R4 and R5 are —CH₂— when R6 is an unbranched —O—C₁-C₅ alkyl.

When the bond between R₂ and R₃ is a double bond, it may be in either the cis-configuration, the trans-configuration, or may consist of a mixture of both cis- and trans-configurations.

Pharmaceutically acceptable salts, esters, amides and acid derivatives of such compounds are further contemplated as within the scope of the compounds of the invention.

Pharmaceutically acceptable salts include base salts such as ammonium salts, alkali metal salts such as sodium and potassium salts, alkaline earth metal salts such as calcium and magnesium salts of Compound 1, organic base salts of Compound 1 such as dicyclohexylamine and N-methyl-D-glucamine salts of Compound 1, and salts of Compound 1 derived by reaction with amino acids such as arginine and lysine. Other pharmaceutically acceptable salts include amine salts of Compound 1, such as methylamine salts, dimethylamine salts, cyclohexylamine salts, benzylamine salts, piperidine salts, ethylenediamine salts, ethanolamine salts, diethanolamine salts, triethanolamine salts, tris(hydroxymethylamino)ethane salts, monomethyl-monoethanolamine salts, procaine salts, caffeine salts and tetraalkyl ammonium salts of Compound 1.

Pharmaceutically acceptable esters of Compound 1 are also contemplated within the scope of the compounds of the invention. In fact, while not being limited by theory, it is believed that such ester derivatives may increase bioavailability of the compounds of the invention by inhibiting β-oxidation and preventing esterification into membrane lipids. Pharmaceutically acceptable esters include lower alkyl esters such as methyl esters, ethyl esters, n-propyl esters, isopropyl esters, n-butyl esters, isobutyl esters, t-butyl esters, pentyl esters and 1-cyclopropylethyl esters; lower alkenyl esters such as vinyl esters and allyl esters; lower alkynyl esters such as ethynyl esters and propynyl esters; hydroxy (lower) alkyl esters such as hydroxyethyl esters; lower alkoxy (lower) alkyl esters such as methoxymethyl esters and 1-methoxyethyl esters; and optionally substituted aryl esters such as, for example, phenyl esters, tosyl esters, t-butylphenyl esters, salicyl esters, 3,4-di-methoxyphenyl esters and benzamidophenyl esters; and aryl (lower) alkyl ester such as benzyl esters, trityl esters and benzhydryl esters. Glycerol and polyethylene glycol esters are also identified herein as pharmaceutically acceptable esters of the compounds of the invention.

Pharmaceutically acceptable acid derivatives of Compound 1 are also contemplated within the scope of the compounds of the invention. In fact, while not being limited by theory, it is believed that such acid derivatives may increase bioavailability of the compounds of the invention by inhibiting β-oxidation and preventing esterification into membrane lipids. Pharmaceutically acceptable acid derivatives include alkyl amides, alkyl amides wherein the nitrogen is optionally bound to one or two C₁₋₄ alkyl groups and amino acid amides. Other useful acid derivatives of the invention include C₁-C₅ alkyl amides, C₁-C₅ alkynyl amides, and C₁-C₅ alkenyl amides. Additional useful acid derivatives also include hydroxamic acid (—NHOH), acyl-cyanamide (—NHCN), and acylsulfonamide (—CO—NH—SO₂—R′) groups. A particularly useful acid derivative of Compound 1 is the N-methylsulfonimide derivative (—NHSO₂CH₃; described in Gauthier et al., Hypertension 42:555, 2003). While again not being limited by theory, it is believed that sulfonimide acid derivatives, in addition to increasing bioavailability as described above, may be particularly useful in the practice of this invention as their pKa values are similar to those of the carboxylic acid groups that they displace.

It is further contemplated that the R₁ group may be one of a variety of groups identified as isosteric replacements of carboxylic acid. One such set of isosteric replacements are 5-substituted-1H-tetrazoles (reviewed in Herr, R. J. Bioorg Med Chem. 2002 November; 10(11):3379-93). A particularly preferred type of isosteric replacement group is 5-substituted-2,4-thiazolidinedione (Boschelli, Diane H.; Connor, David T.; Kuipers, Paul J.; Wright, Clifford D.) Synthesis and cyclooxygenase and 5-lipoxygenase inhibitory activity of some thiazolidin-4-one analogs of meclofenamic acid. Bioorganic & Medicinal Chemistry Letters (1992), 2(7), 705-8). Other isosteric replacements of carboxylic acid that are suitable as the R₁ group include other tetrazoles, oxo-oxadiazoles, oxo-thiadiazoles, mercaptoazoles, sulfinylazoles, sulfonylazoles, isoxazoles, isothiazoles, hydroxy-thiadiazole, hydroxy-chromones, phosphinates, phosphonates, phosphonamides, sulphonates, sulphonamides, and acyl-sulphonamides. The use of such isosteric replacements may result in improved potency, bioavailability, or selectivity of the compounds of the invention. The use and synthesis of various carboxylic acid isosteric replacements in pharmaceutical compounds is described by Wermuth (Wermuth, C. G. “Molecular Variations Based on Isosteric Replacements” in The Practice of Medicinal Chemistry, 2^{nd} Edition, 2003, pp. 189-214).

One exemplary compound of the invention is 13-heptyloxytridec-5(Z)-enoic acid or 13-HTEC. This compound is represented by the structure following immediately below:

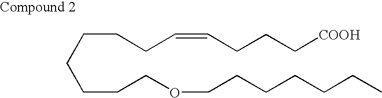

The pharmaceutically acceptable salts, esters, and acid derivatives of 13-HTEC, as described more generally in the preceding section with respect to the generalized formula identified as Compound 1 are thus also contemplated by the instant invention. In particular, C₁-C₅ alkyl ester, —C₁-C₅ alkynyl ester, C₁-C₅ alkenyl ester, glycerol ester, polyethylene glycol ester, C₁-C₅ alkyl amide, C₁-C₅ alkynyl amide, C₁-C₅ alkenyl amide, amino acid amide, hydroxamic acid (—NHOH), acyl-cyanamide (—NHCN), acylsulfonamide (—CO—NH—SO₂—R′), and methylsulfonimide derivatives of the free carboxylic acid group of 13-HTEC are also contemplated as being useful in the practice of the instant invention.

Another exemplary compound of the invention is 14-(hexyloxy)tetradec-5-ynoic acid or 14-HTYC. This compound is represented by the structure:

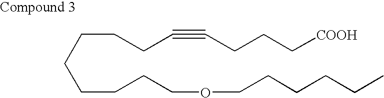

The pharmaceutically acceptable salts, esters, and acid derivatives of 14-HTYC, as described more generally in the preceding section with respect to the generalized formula identified as Compound 1 are thus also contemplated by the instant invention. In particular, C₁-C₅ alkyl ester, —C₁-C₅ alkynyl ester, C₁-C₅ alkenyl ester, glycerol ester, polyethylene glycol ester, C₁-C₅ alkyl amide, C₁-C₅ alkynyl amide, C₁-C₅ alkenyl amide, amino acid amide, hydroxamic acid (—NHOH), acyl-cyanamide (—NHCN), acylsulfonamides (—CO—NH—SO₂—R′), and methylsulfonimide derivatives of the free carboxylic acid group of 14-HTYC are contemplated as being useful in the practice of the instant invention.

Compositions and Means of Administration Useful in the Practice of the Invention

Various pharmaceutical compositions that may be used in the present invention, including at least one compound of the invention such as 13-HTEC, 14-HTYC, and further including pharmaceutically acceptable salts, esters, amides, acid derivatives or prodrugs of the compound of the invention. A “pharmaceutically acceptable derivative or prodrug” means any pharmaceutically acceptable salt, ester, salt of an ester, amides, acid derivative or other derivative of a compound of this invention which, upon administration to a patient, is capable of providing (directly or indirectly) a compound used in this invention.

Such pharmacologic compositions may be formulated in various ways known in the art for administration purposes. To prepare the pharmaceutical compositions of the present invention, an effective amount of the particular compound, in base or acid salt form, as the active ingredient is combined with one or more pharmaceutically-acceptable carriers and delivery vehicles. Numerous pharmaceutically acceptable carriers and delivery vehicles exist that are readily accessible and well-known in the art, which may be employed to generate the preparation desired (i.e., that permit administration of the pharmaceutical composition orally, topically, rectally, percutaneously, by parenteral injection, intranasally or by inhalation). Representative examples of pharmaceutically acceptable carriers and delivery vehicles include aluminum stearate, lecithin, serum proteins, such as human serum albumin; buffer substances such as the various phosphates, glycine, sorbic acid, potassium sorbate, partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica, magnesium trisilicate, polyvinyl pyrrolidone, cellulose-based polymers, polyethylene glycol, sodium carboxymethylcellulose, polyarylates, waxes, polyethylene, polyoxypropylene-block polymers, polyethylene glycol and wool fat, and the like. Cellulose-based polymers include microcrystalline cellulose; methylcellulose; hydroxypropylcellulose; hydroxypropylmethylcellulose; ethylmethylcellulose; carboxymethylcellulose.

The pharmacologic compositions described herein may further be prepared in unitary dosage form suitable for administration orally, percutaneously, by parenteral injection (including subcutaneous, intramuscular, intravenous and intradermal), topically, intranasally, by inhalation, or for application to a medical device, such as an implant, catheter, or other device. In preparing the compositions that permit administration of an oral dosage, for example, any of the pharmaceutically acceptable carriers known in the art may be used, such as water, glycols, oils, alcohols and the like in the case of carriers that permit oral delivery of liquid preparations such as suspensions, syrups, elixirs and solutions. When solid pharmaceutically acceptable carriers are desired that permit oral or rectal administration, starches, sugars, kaolin, lubricants, binders, cellulose and its derivatives, and disintegrating agents and the like may be used to prepare, for example, powders, pills, capsules and tablets.

For pharmaceutically acceptable carriers that permit parenteral administration, the pharmaceutically acceptable carriers often comprise sterile water, which may be supplemented with various solutes or carriers to, for example, increase solubility. Injectable solutions may be prepared in which the pharmaceutically acceptable carrier comprises saline solution, glucose solution, or a mixture thereof, which may include certain well-known anti-oxidants, buffers, bacteriostats, and other solutes or carriers that render the formulation isotonic with the blood of the intended patient, stabilize the composition, and provide for solubilization of the compounds of the invention. To formulate appropriate compositions for parenteral administration of the compounds of the invention, it may also be useful to include one or more of sugars such as mannitol, sorbitol, glucose, or sucrose, glycine, sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; sodium stearoyl-2-actylate; calcium stearoyl-2-lactylate; stearoyl tartrate; sorbitan monostearate; sorbitan tristearate; sorbitan monolaurate; sorbitan monooleate; and/or sorbitan monopalmitate in the final composition.

For pharmaceutically acceptable carriers that permit intranasal administration, the pharmaceutically acceptable carriers often comprise poly acrylic acids such as Carbopol® 940, a hydrogenated castor oil such as Cremophor® RH40, glycerol, vinylpyrrolidones such as PVP-K90® or PVP K30®, polyethylene glycols such as PEG 1450®, benzyl alcohol, Edetate sodium, hydroxycellulose, potassium chloride, potassium phosphate, and sodium phosphate. Compositions used for intranasal administration also commonly include benzalkonium chloride as an anti-microbial preservative.

For pharmaceutically acceptable carriers that permit administration by inhalation, the pharmaceutically acceptable carriers often comprise solvent/carrier/water mixtures that are easily dispersed and inhaled via a nebulizer or inhaler. For example, a mixture of ethanol/propylene glycol/water in the ratio of about 85:10:5 (parts ethanol: parts propylene glycol: parts water) can be used to administer the compounds and compositions of the invention via inhalation. Potential materials that can be used in similar solvent/carrier/water mixtures suitable for compounds of this invention include alcohols, such as ethanol, isopropanol, glycols such as propylene glycol, polyethylene glycol, polypropylene glycol, glycol ether, glycerol and polyoxyethylene alcohols. To suspend or emulsify the compound of the invention can also be useful to include one or more of any of the following materials in the composition: polyoxyethylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; lecithins; alginic acid; sodium alginate; potassium alginate; ammonium alginate; calcium alginate; propane-1,2-diol alginate; agar; carrageenan; locust bean gum; guar gum; tragacanth; acacia; xanthan gum; karaya gum; pectin; amidated pectin; ammonium phosphatides; microcrystalline cellulose; methylcellulose; hydroxypropylcellulose; hydroxypropylmethylcellulose; ethylmethylcellulose; carboxymethylcellulose; sodium, potassium and calcium salts of fatty acids; mono- and di-glycerides of fatty acids; acetic acid esters of mono- and di-glycerides of fatty acids; lactic acid esters of mono- and di-glycerides of fatty acids; citric acid esters of mono- and di-glycerides of fatty acids; tartaric acid esters of mono- and di-glycerides of fatty acids; mono- and diacetyltartaric acid esters of mono- and di-glycerides of fatty acids; mixed acetic and tartaric acid esters of mono- and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; calcium stearoyl-2-lactylate; stearoyl tartrate; sorbitan monostearate; sorbitan tristearate; sorbitan monolaurate; sorbitan monooleate; sorbitan monopalmitate; extract of quillaia; polyglycerol esters of dimerised fatty acids of soya bean oil; oxidatively polymerised soya bean oil; and pectin extract. Compositions useful for administration of compounds by inhalation, including various buffers, preservatives, bacteriocides and bacteriostats are described in U.S. Patent Application No. 20040265238, incorporated herein by reference in its entirety.

Administration is achieved with a nebulizer or inhaler that forms droplets from a solution or liquid containing the active compounds of the invention. Nebulizers useful in administering the active compounds of the invention include jet nebulizers such as the MiniHEART™ (Westmed, Tucson, Ariz.) and AM-601 Medicator Aerosol Delivery System™. (Healthline Medical, Baldwin Park, Calif.) systems. Ultrasonic nebulizers such as the MabisMist™ Ultrasonic Nebulizer (MABIS Healthcare, Waukegan, Ill.) may also be used to administer the compounds and compositions of the invention.

Alternatively, solid formulations, usually in the form of a powder, may be inhaled in accordance with the present invention. In such cases, the particles are preferably less than 10 micrometers in diameter, and more preferably, less than 5 micrometers in diameter. Most preferable are formulations that result in a particles that are less than 1 micrometers in diameter (Bayat, M., and Cook, A. M. J. Neurosci. Nursing 36(4):231, 2004). Dry powder inhalers are typically used to administer solid formulations. Metered dose inhalers may also be used to administer solid formulations. Various pharmaceutically acceptable propellants may also be useful in administering solid formulations of the compounds or compositions of the invention.

In summary, those skilled in the art will recognize that a variety of formulations and administration techniques can be used to aerosolize the compounds and/or compositions of the invention. A useful summary of formulations and delivery methods suitable for aerosolized delivery of therapeutic agents such as those described herein is found in U.S. Patent Application No. 20040265238, incorporated herein by reference in its entirety.

Methods of Treating Pulmonary Hypertension

Individuals skilled in the art will further recognize that the compounds of the invention as well as compositions comprising at least one compound of the invention will be useful in decreasing pulmonary hypertension in subjects in need thereof. A variety of observations can be made to identify subjects benefiting from reductions in pulmonary hypertension. At a symptomatic level, subjects in need thereof may be identified as individuals experiencing breathlessness, fainting, fatigue, swelling of the ankles and legs, dizziness, or chest pain. More definitive tests for pulmonary and/or cardiac function can also be performed to confirm a diagnosis of pulmonary hypertension and identify subjects in need. Tests for pulmonary and cardiac function include but are not limited to various exercise based tests such as the six minute walk test, or other testing procedures such as echocardiograms, electrocardiograms, right heart catheterization, and pulmonary function tests. Pulmonary function tests that can be also be used to both identify subjects in need and to assess the therapeutic effects of the methods described herein include any method or test for determining pulmonary arterial pressure (PPA), left atrial pressure (PLA), central venous pressure (PCV), systemic arterial pressure (PSYS), heart rate (HR), cardiac output (CO), pulmonary artery wedge pressure (Ppa,we), right ventricular ejection fraction (RVEF), and central venous oxygen saturation (Sv_{O2}). Techniques for obtaining and using these measurements to both identify subjects with pulmonary hypertension and to assess the efficacy of pulmonary hypertension treatments are well known to those skilled in the art (Olschewski, et al. Am. J. Respir. Crit. Care Med. 160(2): 600-607, 1999; and U.S. Pat. No. 6,756,033, herewith incorporated by reference in its entirety). In general, subjects in need can be identified by obtaining pulmonary function data such as PPA, PLA, PCV, PSYS, HR, CO, Ppa,we, RVEF, and/or Sv_{O2} and comparing the values obtained to normal range of values for healthy individuals of similar age, sex and weight who live at the same altitude and are engaged in similar activities. Similarly, the efficacy of the method in reducing pulmonary hypertension can likewise be assessed by collecting data such as PPA, PLA, PCV, PSYS, HR, CO, Ppa,we, RVEF, and/or Sv_{O2} from subjects following or in the course of being administered the compounds or compositions of the invention and comparing the values obtained to the values observed prior to administration of the compounds or compositions of the invention.

Administration can be achieved by methods including but not limited to oral, topical, rectal, percutaneous, parenteral injection, intranasal or inhalation-based delivery techniques. In addition to direct injection based techniques of parenteral delivery, the use of pumps that permit regulated delivery of the compounds and compositions of the invention is also contemplated. For example, portable infusion pumps such as the CADD-PCA, CADD-Plus, CADD-1, CADD-Legacy 6400 and 6500 (Smiths Medical MD, Inc., St. Paul, Minn., USA) can also be used to deliver the compounds and compositions of the invention via an implanted catheter placed in a central vein of the subject.

Inhalation based techniques of delivery can be through use of a nebulizer or inhaler that forms droplets from a solution or liquid containing the active compounds of the invention. Nebulizers useful in administering the active compounds of the invention include jet nebulizers such as the MiniHEART™ (Westmed, Tucson, Ariz.) and AM-601 Medicator Aerosol Delivery System.™ (Healthline Medical, Baldwin Park, Calif.) systems. Ultrasonic nebulizers such as the MabisMist™ Ultrasonic Nebulizer (MABIS Healthcare, Waukegan, Ill.). Dry powder inhalers are typically used to administer solid formulations by inhalation. Metered dose inhalers may also be used to administer solid formulations. The inhalation-based delivery device may include a pharmaceutically acceptable propellant for facilitating delivery of the compounds or compositions of the invention.

In summary, those skilled in the art will recognize that a variety of formulations and administration techniques can be used to aerosolize the compounds and/or compositions of the invention. A useful summary of inhalation-based administration methods generally applicable to the aerosolized delivery of the compounds and compositions of the invention is found in U.S. Patent Application No. 20040265238, incorporated herein by reference in its entirety.

Appropriate doses of the compounds or compositions of the invention that decrease in pulmonary hypertension in a subject can be determined empirically by any number of methods familiar to those skilled in the art. For example, baseline measurements of pulmonary function data such as PPA, PLA, PCV, PSYS, HR, CO, Ppa,we, RVEF, and/or Sv_{O2} of test subjects can be recorded prior to dosing with known amounts of the compound or composition of the invention. At an appropriate interval following dosing, the pulmonary function data such as PPA, PLA, PCV, PSYS, HR, CO, Ppa,we, RVEF, and/or Sv_{O2} of test subjects can be determined and compared to the pre-dosing values. An appropriate dose can then be determined based on the magnitude of the observed effect on the pulmonary function data. It is understood by one skilled in the art that other test subjects that receive placebos that omit the compounds of the invention may also be useful in determining doses by empirical tests. Finally, it is further understood that empirical dosing tests such as those described above can first be conducted in subjects such as rats or rabbits to obtain rough estimates of the appropriate dosing rate (i.e., amount of the compound of the invention per weight of subject; typically in mg per kg or μg per kg). This empirically determined dose rate obtained in a test subject such as a rat or a rabbit can then be extrapolated to other subjects such as a sheep, a pig, a horse, a cow or a human by simply administering the compound of the invention to the other subject at approximately the same dose rate that was empirically determined in the test subject. It is anticipated that iterative rounds of such dosage optimization tests as described here will be conducted to determine a dose rate that is appropriate for each subject that may be a rabbit, a sheep, a pig, a rat, a horse, a cow or a human.

The specific therapeutically effective dose rate that results in a decrease in pulmonary hypertension for any particular patient may depend upon a variety of factors, including the level of disease severity, the level of compound activity within a given patient; the activity of the specific compound employed; the specific pharmacologic formulation employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts. Furthermore, it may be appropriate to administer the required dose more than once in a twenty-four hour period, such as for example in two, three, four or more sub-doses at appropriate intervals throughout the day. It is also anticipated that the same empirical testing method described previously for determining the dose rate can similarly be employed to empirically determine optimal intervals for administering sub doses that result in decreased pulmonary hypertension.

By way of example only, the total daily dose of one or more of the compounds of the invention may be provided to a patient in single or in divided doses, which may be in amounts from about 0.01 to 100 mg/kg body weight. Single dose compositions may contain such amounts or submultiples thereof to make up the daily dose. As described above, treatment regimens according to the present invention may comprise administering to a patient an appropriate daily total dose each day in single or multiple sub doses. A variety of references are cited herein. The contents of each of these references is herein incorporated by reference in their entirety.

### EXAMPLES

The following examples illustrate the methods of modulating pulmonary hypertension as well as methods of preparing compounds that are useful in modulating pulmonary hypertension. The examples demonstrate certain methods and are not intended to limit the scope of the present invention. Those of skill in the art should, in light of the present disclosure, appreciate that many changes can be made in the specific embodiments which are disclosed and still obtain a like or similar result without departing from the spirit and scope of the invention.

### Example 1

To prepare 5,6-EET, selective epoxidation of arachidonic acid was achieved using the method of Corey (Corey E. J., et al. J Am Chem Soc 101: 1586-1587, 1979) as described previously (Stephenson A. H., et al., Am J Physiol 275: H100-H109, 1998). In its free-acid form, 5,6-EET readily decomposes to 5,6-dihydroxyeicosatrienoic acid (DHET) and the corresponding δ-lactone. Therefore, before use, 5,6-EET was re-purified by reverse-phase HPLC. The structure of 5,6-EET is as shown below:

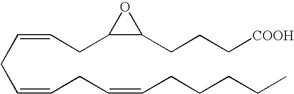

To prepare 14,15-epoxyeicosa-5(Z)-enoic acid (14,15-EEZE) the procedures described in Falck J. R., et al., Am J. Physiol. 2003; 284: H337-H349 can be employed.

To prepare methylsulfonimide derivatives of compounds of the invention, procedures described in Falck J. R., et al., Am J Physiol. 2003; 284: H337-H349 to synthesize the methylsulfonimide derivative of 14,15-EEZE can be employed to synthesize similar methylsulfonimide derivatives of the compounds of the invention. In brief, the carboxylic acid of the compound of the invention and N-hydroxysuccinimide (NHS) can be mixed and azeotropically dried with anhydrous benzene in similar molar ratios to those described by Falck, J. R. et al. (ibid). The mixture can then be dissolved in dry THF to which 1,3-dicyclohexylcarbodiimide (DCC) is added in similar molar ratios to those described by Falck, J. R. et al. (ibid). After being stirred at room temperature for 12 h, all volatiles are removed in vacuo and the residue purified by SiO₂ column chromatography to give the hydroxysuccinimide ester (NHS ester). The NHS ester is dissolved in dry HMPA to which N,N-dimethylaminopyridine and methanesulfonamide are added in the ratios indicated by Falck et al. (ibid.). After heating for 2 h at 90 C, the reaction mixture was purified to give the methylsulfonamide derivative (75% yield).

### Example 2

The ability of the exemplary compounds 13-heptyloxytridec-5(Z)-enoic acid (13-HTEC) and 14-(hexyloxy)tetradec-5-ynoic acid (14-HTYC) to specifically inhibit 5,6-EET induced vasoconstriction of pulmonary arteries was demonstrated in rabbit intralobar pulmonary arteries. To obtain the pulmonary arteries used in the study, adult New Zealand white rabbits (2.4-3.0 kg) were anesthetized with pentobarbital sodium (15 mg/kg iv) for 10 minutes after intramuscular administration of ketamine (8 mg/kg) and xylazine (2 mg/kg). A tracheostomy was performed and a tracheal cannula was inserted. The animals were ventilated via a fixed volume ventilator (Harvard) with room air (tidal volume: 8-10 ml/kg at 15 cycles/min). A catheter was inserted into a carotid artery for administration of heparin (1,000 units, iv) 10 minutes before exsanguination of the animal. After exsanguination, the lungs were removed for isolation of the pulmonary vessels. Intralobar, second order pulmonary arteries (PA) were obtained as described previously (Stephenson A. H., et al., American Journal of Physiology 284: H2153-H2161, 2003). Briefly, intralobar PA (1-2 mm OD) were dissected free of extravascular tissue, cut into rings 3-4 mm in length and suspended in water-jacketed tissue chambers containing 10 ml of a physiological salt solution (PSS) composed of (in mM): NaCl (118.3), KCl (4.7), CaCl₂ (2.5), MgS0₄ (1.2), KH₂P0₄ (1.2), NaHC0₃ (25) Na-EDTA (0.026), and glucose (11.1). The PSS was gassed with 95% O₂-5% CO₂ (pH: 7.4) and maintained at 37° C. Each ring was mounted between two stainless steel support wires. Ring tension was measured from one of the support wires attached to an isometric force transducer (FT03, Grass) and recorded continuously on a polygraph (Grass). Each ring was placed under a basal (passive) tension (0.75 to 1.5 g) determined to result in a maximal contractile response to KCl (60 mM). In some studies, basal tension was increased with 25 mM KCl to achieve a submaximal increase in active tension. At basal tension, concentrations of 5,6-EET (1×10⁻⁸-1×10⁻⁵ M, delivered in 1 μl of absolute ethanol) were added individually or cumulatively to the rings in 10 ml PSS. Total ethanol concentration in the tissue chambers never exceeded 0.1%. In these experiments, 5,6-EET was added in the presence or absence of the selective 5,6-EET inhibitors 13-HTEC (10 μM) and 14-HTYC (10 μM). At the concentrations used, the ethanol vehicle for added agents did not alter the basal tension or active tension of the PA rings. PA rings were also exposed to various concentrations of PGF_{2α}, (Sigma Chemical Company, St. Louis, Mo.), 14,15-EET (Cayman Chemical, Ann Arbor, Mich.), 5-hydroxytryptamine (5-HT) (Sigma Chemical Company, St. Louis, Mo.), and norepinephrine (NE) (Sigma Chemical Company, St. Louis, Mo.).

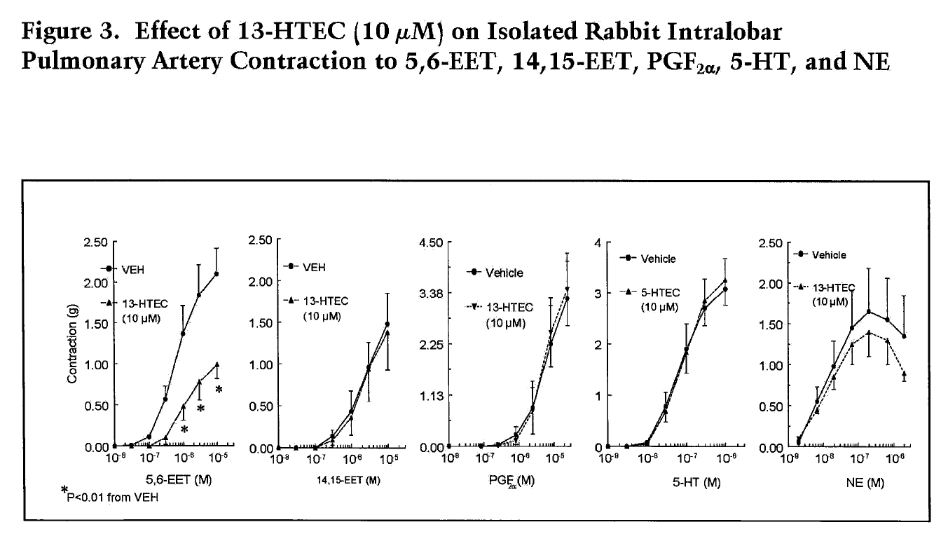

FIG. 3 illustrates the results of exposing the rabbit pulmonary artery sections to 13-HTEC in the presence of various concentrations of 5,6-EET, PGF_{2α}, 14,15-EET, 5-hydroxytryptamine (5-HT) and norepinephrine (NE). The exemplary compound 13-HTEC at 10 μM significantly inhibited the vasoconstrictor activity of exogenously administered 5,6-EET, shifting the concentration-response curve to the right. Similar inhibition of 5,6-EET-mediated vasoconstriction is also observed when the PA sections are treated with the structurally related analog 14-HTEC. However, 13-HTEC does not inhibit PGF_{2α}, 14,15-EET, and 5-hydroxytryptamine (5-HT)-mediated vasoconstriction of PA, indicating that the inhibitory action of 13-HTEC is specific. The compound 13-HTEC may have partially inhibited norepinephrine (NE)-mediated vasoconstriction of PA. However, vasoconstriction of PA mediated by an adrenergic agonist similar to NE has been reported previously to be partially dependent on the vasoconstrictor activity of EETs (Zhu et al., American Journal of Physiology—Lung Cellular & Molecular Physiology 278: L335-L343, 2000).

### Example 3

The ability of the exemplary compound 13-heptyloxytridec-5(Z)-enoic acid (13-HTEC) to specifically inhibit hypoxia-induced pulmonary vasoconstriction was also demonstrated in isolated perfused rabbit lungs. Adult New Zealand white rabbits were prepared as described in Example 2. After exsanguination was completed, a midsternal thoracotomy was performed, and the heart and lungs were removed en bloc. Fluid-filled catheters were placed into the PA and the left atrium for lung perfusion and pressure measurements. The isolated lungs were ventilated at 10 ml/kg with 26-30 breaths/min of 15% O₂-6% CO2-79% N₂ to achieve a perfusate pH of 7.33±0.01, PCO₂ of 38.1±2.8 mmHg, and PO₂ of 107.3±5.9 mmHg. The lungs were perfused in a humidified chamber (34-37° C.) in a recirculating manner with 150 ml of PSS containing (in mM) 118.3 NaCl, 4.7 KCl, 2.5 CaCl2, 1.2 MgSO4, 1.2 KH2PO4, 25.0 NaHCO3, 0.026 Na-EDTA, and 11.1 glucose (pH 7.4) to which hetastarch was added for maintenance of oncotic pressure. The Nitric Oxide synthesis inhibitor L-NAME (1-nitro-arginine methyl ester; Sigma Chemical Company, St. Louis, Mo.) was also added 30 minutes before hypoxia was initiated at a concentration of 100 μM to inhibit endogenous nitric oxide synthase, prevent nitric oxide-induced pulmonary vasodilation and therefore, to obtain a strong hypoxia-induced pulmonary vasoconstriction response (V. L. Brashers, M. J. Peach, and C. E. Rose Jr, Journal of Clinical Investigation, November 1988; 82(5): 1495-502). Pulmonary arterial (Ppa, inflow), pulmonary venous (Pla, outflow), and airway pressures were recorded continuously. Microvascular pressure (Pmv) was measured by the double occlusion method (Dawson C. A., et al., Journal of Applied Physiology 64: 274-284, 1988; Wurtz M. M., et al., J Appl Physiol 73: 2135-2141, 1992). To prevent atelectasis, positive end-expiratory airway pressure (1-1.5 mmHg) was maintained throughout each experiment. Lungs were perfused under zone III conditions (Ppa>Pla>airway pressure) with a roller pump (Masterflex™, Cole Parmer Instrument, Vernon Hills, Ill.) at 100 ml/min. Outflow pressure was adjusted to 2-3 mmHg via a screw clamp on the outflow tubing. In this experiment, 13-HTEC at 1 or 10 μM was added to the perfusate reservoir 15 min before measurements of vascular pressures were obtained. Total PVR was calculated as (Ppa Pla)/perfusate flow rate. Arterial PVR was calculated as (Ppa Pmv)/perfusate flow rate, and venous PVR was calculated as (Pmv Pla)/perfusate flow rate (Stephenson A. H., et al., Am J Physiol 275: H100-H109, 1998).

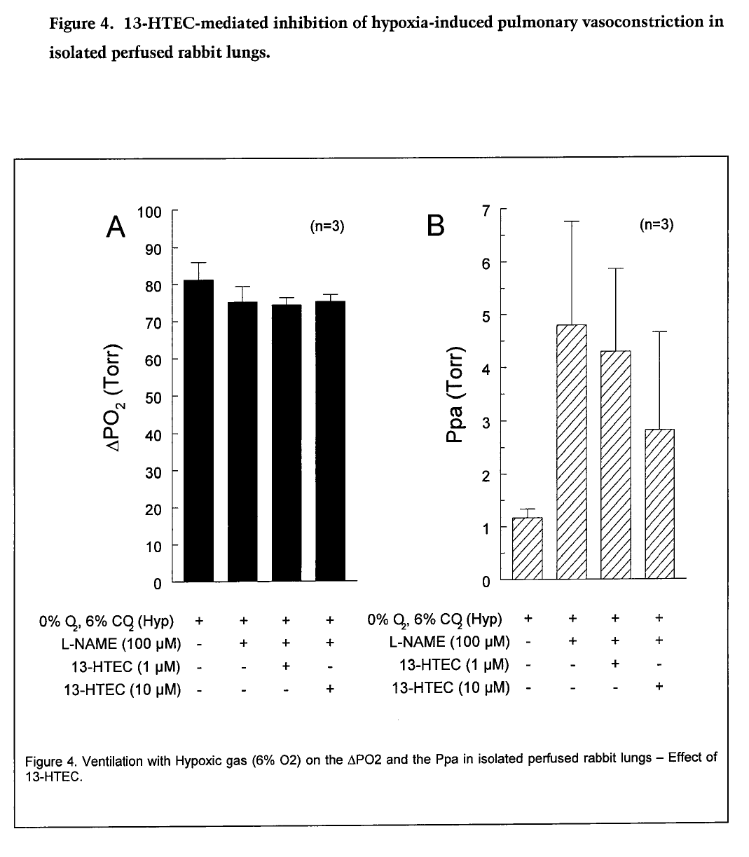

FIG. 4 illustrates the results of the exposing the perfused rabbit lungs to 13-HTEC under conditions that promote hypoxia induced pulmonary vasoconstriction. As shown in the right-most column of Panel B in FIG. 4, 13-HTEC at (10 μM) significantly decreased Pulmonary arterial inflow (Ppa) pressure. Pulmonary arterial inflow pressure (Ppa) is directly related to the degree of vasoconstriction such that decreased Ppa is indicative of decreased pulmonary vasoconstriction. It consequently follows that administration of effective doses of 13-HTEC at (10 μM) are expected to result in decreased pulmonary hypertension resulting form exposure to hypoxia.

### Example 4

The ability of the exemplary compound 14,15-epoxyeicosa-5(Z)-enoic acid (14,15-EEZE) to promote or agonize 5,6-EET induced vasoconstriction of pulmonary arteries was demonstrated in rabbit intralobar pulmonary arteries. Adult New Zealand white rabbits (2.4-3.0 kg) were obtained and prepared as described in Example 2. Intralobar, second order pulmonary arteries (PA) were also obtained as described previously in Example 2 and as in Stephenson A. H., et al., American Journal of Physiology 284: H2153-H2161, 2003). In these experiments, 5,6-EET was added in the presence or absence of 14,15-EEZE (1 μM). At the concentrations of 14-15-EEZE used (1 and 10 μM), the ethanol vehicle (0.01%) for added agents did not alter the basal tension or active tension of the PA rings.

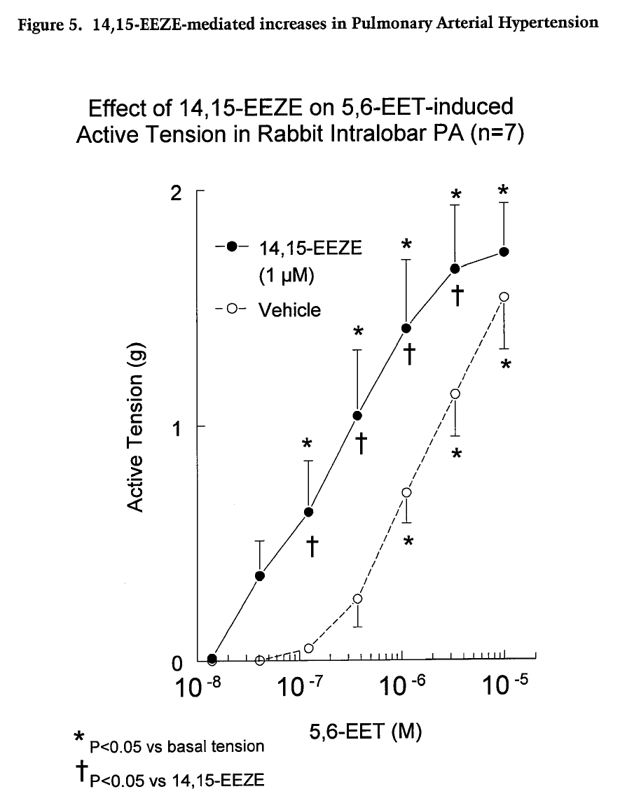

FIG. 5 illustrates the results of the exposing the rabbit pulmonary artery sections to 14,15-EEZE. The exemplary compound 14,15-EEZE at 1 μM significantly promoted or agonized the vasoconstrictor activity of exogenously administered 5,6-EET, shifting the concentration-response curve to the left (i.e., pulmonary arterial pressure is increased by 14,15-EEZE at lower concentrations of 5,6-EET relative to the vehicle controls). The administration of 14,15-EEZE under conditions where enhanced pulmonary vasoconstriction is desirable, such as in pathological conditions associated with inflammation, such as, but not limited to, pneumonia, is thus contemplated. 14,15-EEZE and its related analogs are also desirable in treatment of sepsis or Adult Respiratory Distress Syndrome (ARDS).

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

### Example 5

Synthesis of 13-heptyloxytridec-5(Z)-enoic acid (13-HTEC) was achieved as follows. In general, ¹H NMR spectra were recorded using a Varian (Palo Alto, Calif., USA) Mercury (300 MHz) or Varian INOVA (400 MHz) in CDCl₃ with tetramethylsilane (δ0.00) as internal standard. ¹³C NMR spectra were recorded in CDCl₃ using a Varian Mercury (75 MHz) or Varian INOVA (100 MHz) with CDCl₃ (δ77.23) as an internal standard. Abbreviations are s, singlet; d, doublet; t, triplet; q, quartet; m, multiplet; and br, broad. Data were presented as chemical shift, multiplicity, integration and coupling constant. Analytical thin layer chromatography was carried out on Kieselgel 60 F₂₅₄ plates (EM Science, Cherry Hill, N.J., USA) with visualization by UV (254 nm) and anisaldehyde or phosphomolybdic acid solutions. Purification of products was performed by column chromatography on silica gel (EM Science, Silica gel 60, 63-200 μm). Reagents and solvents were purified by standard means. CH₂Cl₂ was distilled from CaH₂; THF and Et₂O were distilled from Na/benzophenone ketyl.

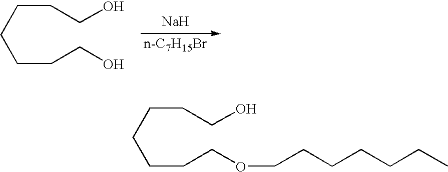

To a 0° C. solution of 1,7-heptanediol (9.24 g, 70 mmol) in dry DMF (30 mL) was added NaH (55% oil suspension, 1.22 g, 28 mmol) in portions (ca. 8) over a period of 15 min. Stirring was continued at 0° C. for 30 min, then at room temperature for another 30 minutes. The reaction mixture was then cooled to −10° C. and a solution of 1-bromoheptane (2.51 g, 14 mmol) in dry DMF (10 mL) was added. After stirring at room temperature, it was cooled to 0° C., quenched with ice, and extracted with ether (3×100 mL). The combined ethereal extracts were washed with brine (1×100 mL), dried (Na₂SO₄), and concentrated in vacuo. The residue was purified by SiO₂ column chromatography to give 7-heptyloxyheptan-1-ol (2.5 g, 77%). TLC: 20% EtOAc/hexanes, R_{f}=0.21, charred with anisaldehyde. ¹H NMR (CDCl₃, 400 MHz) δ 0.87 (t, 3H, J=6.8 Hz), 1.14-1.23 (m, 14H), 1.50-1.62 (m, 6H), 3.38 (t, 4H, J=6.8 Hz), 3.62 (t, 2H, J=6.7 Hz).

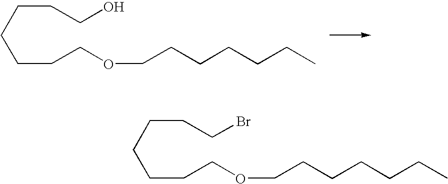

Carbon tetrabromide (4.87 g, 14.67 mmol) was added to a 0° C. solution of the above alcohol (2.5 g, 10.87 mmol) in dry dichloromethane (20 mL) under an argon atmosphere followed by the slow (30 min) introduction of a solution of triphenylphosphine (4.30 g, 16.31 mmol) in dry dichloromethane (20 mL). After 30 min, all volatiles were removed in vacuo and the residue was filtered through a bed of silica gel eluting with 5% ether/hexanes to give 1-bromo-7-(heptyloxy)heptane (3.0 g, 94%). TLC: 20% EtOAc/hexanes, R_{f}=0.82, charred with anisaldehyde. ¹H NMR (CDCl₃, 400 MHz) δ 0.87 (t, 3H, J=7.2 Hz), 1.20-1.48 (m, 14H), 1.50-1.62 (m, 4H), 1.80-1.90 (m, 2H), 3.34-3.44 (m, 6H).

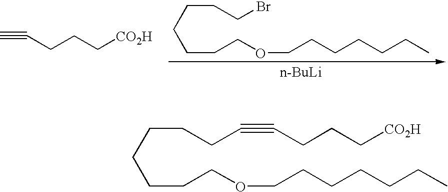

n-BuLi (2.5 M soln in hexanes, 5.4 mL, 13.64 mmol) was added dropwise to a −40° C. solution of 5-hexynoic acid (0.78 g, 6.82 mmol) in dry THF/HMPA (20 mL, 4:1). After stirring for 4 h at −40° C., a solution of the above bromide (2 g, 6.82 mmol) in THF (10 mL) was added at the same temperature and the reaction mixture was gradually warmed to room temperature. After 12 h, the reaction mixture was acidified with aq. 1M oxalic acid, extracted into ether, and the combined ethereal extracts were dried and concentrated in vacuo. The residue was purified by SiO₂ column chromatography to give 13-heptyloxytridec-5-ynoic acid (1.55 g, 71%). TLC: 50% EtOAc/hexanes, R_{f}=0.74, charred with anisaldehyde. ¹H NMR (CDCl₃, 400 MHz) δ 0.87 (t, 3H, J=7.0 Hz), 1.20-1.65 (m, 20H), 1.80 (quintet, 2H, J=7.1 Hz), 2.08-2.18 (m, 2H), 2.20-2.28 (m, 2H), 2.49 (t, 2H, J=7.2 Hz), 3.40 (t, 4H, J=6.8 Hz).

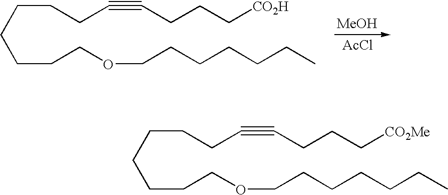

Acetyl chloride (2 mL, 28.02 mmol) was slowly added to dry MeOH (120 mL) at 0° C. After 2 h at room temperature, a solution of above acid (1.55 g, 4.77 mmol) in MeOH (10 mL) was added. After 12 h, all volatiles were removed in vacuo and the residue was dissolved in ether, washed with 5% aq. sodium bicarbonate, brine, dried, and the residue purified by silica gel chromatography to give methyl 13-heptyloxytridec-5-ynoate (1.58 g, 98%). TLC: 20% EtOAc/hexanes, R_{f}=0.60, charred with anisaldehyde. ¹H NMR (CDCl₃, 400 MHz) δ 0.87 (t, 3H, J=7.2 Hz), 1.20-1.60 (m, 20H), 1.81 (quintet, 2H, J=7.3 Hz), 2.08-2.15 (m, 2H), 2.17-2.24 (m, 2H), 2.43 (t, 2H, J=7.2 Hz), 3.38 (t, 4H, J=6.8 Hz), 3.67 (s, 3H).

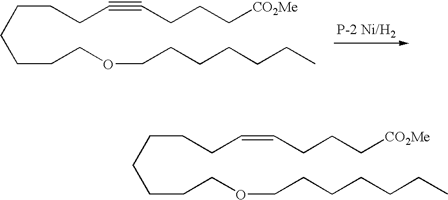

NaBH₄ (44 mg, 1.17 mmol) was added to a stirring, room temperature solution of Ni(OAc)₂.(H₂O)₄ (291 mg, 1.17 mmol) in absolute EtOH (10 mL) under a hydrogen atmosphere (1 atmosphere). After 20 min, freshly distilled ethylenediamine (141 mg, 2.34 mmol) was added and the stirring was continued for another 20 min before a solution of the above acetylenic compound (1.58 g, 4.67 mmol) in absolute ETOH (10 mL) was added. After 2 h, the reaction mixture was filtered over a bed of silica gel eluting with ether. The filtrate was evaporated in vacuo to give methyl 13-heptyloxytridec-5(Z)-enoate (1.50 g, 94%) as a colorless syrup sufficiently pure to be used directly in the next reaction. TLC: 20% EtOAc/hexanes, R_{f}=0.62, charred with anisaldehyde. ¹H NMR (CDCl₃, 400 MHz) δ 0.87 (t, 3H, J=7.2 Hz), 1.22-1.40 (m, 16H), 1.50-1.60 (m, 4H), 1.63-1.72 (m, 2H), 1.94-2.10 (m, 4H), 2.30 (t, 2H, J=7.6 Hz) 3.38 (t, 4H, J=6.8 Hz), 3.66 (s, 3H), 5.25-5.45 (m, 2H); ¹³C NMR (75.5 MHz, CDCl₃) δ 14.19, 22.75, 25.00, 26.33, 26.64, 27.32, 29.33, 29.38, 29.53, 29.76, 29.93, 29.95, 31.99, 33.49, 51.43, 71.01, 71.05, 128.46, 131.17.

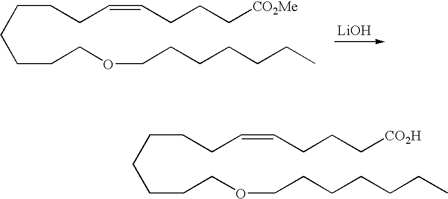

To a solution of the above ester (430 mg, 1.26 mmol) in THF/H₂O (4:1, 40 mL) was added a 1M aq. solution of LiOH (4.2 mL, 4.2 mmol) at 0° C. After stirring at room temperature for 12 h, the reaction mixture was acidified to pH 5 with 1M aqueous oxalic acid solution. All volatiles were removed in vacuo and the residue was dissolved in ethyl acetate, washed with water, brine, dried (Na₂SO₄), and evaporated to give 13-heptyloxytridec-5(Z)-enoic acid (13-HTEC) (400 mg, 97%). TLC 20% EtOAc/hexanes, R_{f}=0.42, charred with anisaldehyde. ¹H NMR (CDCl₃, 400 MHz) δ 0.87 (t, 3H, J=7.2 Hz), 1.20-1.40 (m, 16H), 1.50-1.62 (m, 4H), 1.65-1.75 (m, 2H), 1.95-2.05 (m, 2H), 2.05-2.14 (m, 2H), 2.35 (t, 2H, J=7.6 Hz), 2.40 (t, 2H, J=6.8 Hz), 3.40 (t, 4H, J=6.8 Hz), 5.25-5.46 (m, 2H); ¹³C NMR (100 MHz, CDCl₃) δ 14.29, 22.83, 24.82, 26.34, 26.62, 27.41, 29.38, 29.39, 29.54, 29.78, 29.88, 29.90, 32.05, 33.59, 71.16, 71.19, 128.44, 131.47, 179.81. ESMS m/z 327.2 (M+1).

### Example 6

Synthesis of 14-(hexyloxy)tetradec-5-ynoic acid (14-HTYC) was achieved as follows. In general, ¹H NMR spectra were recorded using a Varian (Palo Alto, Calif., USA) Mercury (300 MHz) or Varian INOVA (400 MHz) in CDCl₃ with tetramethylsilane (δ0.00) as internal standard. ¹³C NMR spectra were recorded in CDCl₃ using a Varian Mercury (75 MHz) or Varian INOVA (100 MHz) with CDCl₃ (δ77.23) as an internal standard. Abbreviations are s, singlet; d, doublet; t, triplet; q, quartet; m, multiplet; and br, broad. Data were presented as chemical shift, multiplicity, integration and coupling constant. Analytical thin layer chromatography was carried out on Kieselgel 60 F₂₅₄ plates (EM Science, Cherry Hill, N.J., USA) with visualization by UV (254 nm) and anisaldehyde or phosphomolybdic acid solutions. Purification of products was performed by column chromatography on silica gel (EM Science, Silica gel 60, 63-200 μm). Reagents and solvents were purified by standard means. CH₂Cl₂ was distilled from CaH₂; THF and Et₂O were distilled from Na/benzophenone ketyl.

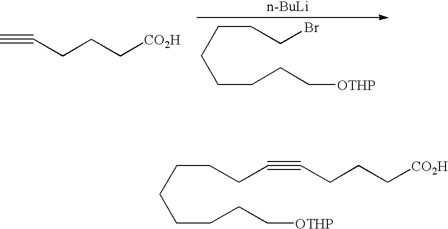

To a solution of 5-hexynoic acid (6.81 g, 60.69 mmol) in dry THF (130 mL) and HMPA (32.60 g, 182 mmol) was added n-BuLi (2.5 M solution in hexanes, 48.5 mL, 121.4 mmol) at −40° C. The reaction mixture was warmed to room temperature over 3 h and kept there for another 1 h. The reaction mixture was re-cooled to −40° C. and a solution of the above bromide (17.80 g, 60.68 mmol, Lancaster) in THF (30 mL) was added. After stirring overnight at room temperature, the reaction was quenched with water and the mixture was washed with ether. The separated aqueous layer was acidified pH 5 with 1M aq. oxalic acid and then extracted with ether. The combined ethereal extracts were dried over Na₂SO₄ and concentrated in vacuo. The residue was purified by silica gel column chromatography to 14-(tetrahydropyran-2-yloxy)tetradec-5-ynoic acid (12.03 g, 80% conversion based on recovery of 4.24 g of the starting bromide. TLC: 50% EtOAc/hexanes, R_{f}=0.55, charred with anisaldehyde. ¹H NMR (CDCl₃, 400 MHz) δ 1.22-1.90 (m, 20H), 2.07-2.16 (m, 2H), 2.20-2.27 (m, 2H), 2.49 (t, 2H, J=7.2 Hz), 3.35-3.44 (m, 1H), 3.45-3.54 (m, 2H) 3.68-3.78 (m, 1H), 3.85-3.96 (m, 1H), 4.53-4.62 (m, 1H).

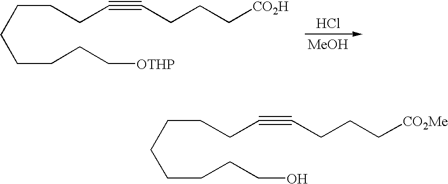

Acetyl chloride (4 mL, 56 mmol) was added slowly to dry MeOH (140 mL) at room temperature. After 2 h, a solution of the above THP-carboxylic acid (2.40 g, 7.40 mmol) in dry MeOH (30 mL) was added at room temperature and stirred overnight. The reaction mixture was cooled to 0° C. and neutralized with Et₃N. All volatiles were evaporated under reduced pressure and the residue was dissolved in ether, filtered, and the ethereal layer was washed with water, brine, dried over Na₂SO₄ and concentrated in vacuo. The residue was purified by silica gel column chromatography eluting in 25% ether/hexanes to give methyl 14-hydroxytradec-5-ynoate (1.67 g, 88%). TLC: 50% EtOAc/hexanes, R_{f}=0.54, charred with anisaldehyde. ¹H NMR (CDCl₃, 400 MHz) δ 1.25-1.60 (m, 12H), 1.82 (quintet, 2H, J=7.1 Hz), 2.08-2.17 (m, 2H), 2.17-2.25 (m, 2H), 2.43 (t, 2H, J=7.2 Hz), 3.63 (t, 2H, J=6.4 Hz), 3.67 (s, 3H).

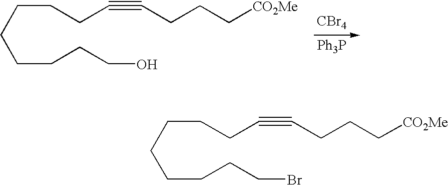

To a 0° C. solution of the above alcohol (1.60 g, 6.30 mmol) in dry CH₂Cl₂ (25 mL) was added CBr₄ (2.82 g, 8.5 mmol) followed by Ph₃P (2.47 g, 9.4 mmol) in portions. After 15 min, the reaction mixture was filtered through a silica gel column eluting with 50% ether/hexanes to give methyl 14-bromotradec-5-ynoate (2.0 g, 100%). TLC: 20% EtOAc/hexanes, R_{f}=0.60, charred with anisaldehyde. ¹H NMR (CDCl₃, 400 MHz) δ 1.24-1.60 (m, 10H), 1.75-1.90 (m, 4H), 2.10-2.16 (m, 2H), 2.18-2.25 (m, 2H), 2.42 (t, 2H, J=7.6 Hz), 3.41 (t, 2H, J=6.8 Hz), 3.67 (s, 3H).

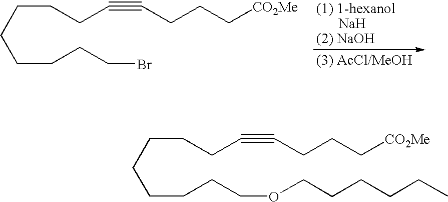

To a 0° C. solution of 1-hexanol (3.2 g, 31.6 mmol) in dry THF (40 mL) under an argon atmosphere was added NaH (55% oil dispersion prewashed with hexanes, 1.37 g, 31.6 mmol). After 1 h, the above bromide (2.0 g, 6.3 mmol) and 15-crown-5 (2.56 g, 11.63 mmol) in THF (30 mL) were added. After 3 h at room temperature, the reaction was acidified to pH 5 with 1M aq. oxalic acid and extracted with ether. The ethereal extracts were washed with brine, dried over Na₂SO₄ and concentrated in vacuo to give an approximate 1:3 mixture of methyl 14-hexyloxytetradec-5-ynoate, n-hexyl 14-hexyloxytetradec-5-ynoate, and unreacted 1-hexanol (2.80 g crude product). TLC: 20% EtOAc/hexanes, R_{f}=0.70 and 0.74, charred with anisaldehyde. The above crude ester mixture and NaOH (2.60 g, 0.065 mmol) were stirred in THF/H₂O (3:1, 45 mL) at room temperature for 12 h and then the THF was evaporated in vacuo. The resultant aqueous solution was washed with ether (3×25 mL), cooled to 0° C., acidified to pH 5 using 1M aq. oxalic acid, and extracted with ether. The residue was used in the following reaction without further purification.

Acetyl chloride (4 mL, 56 mmol) was carefully added to dry MeOH (140 mL) at 0° C. After stirring at room temperature for 1 h, a solution of the above crude carboxylic acid in dry MeOH (40 mL) was added and the mixture was warmed to room temperature. After stirring overnight, most of the MeOH was removed in vacuo and the residue was neutralized with 5% aqueous sodium bicarbonate and extracted with ether. The combined ethereal extracts were washed with brine, dried over Na₂SO₄ and concentrated in vacuo. The residue was purified by silica gel column chromatography to give methyl 14-(hexyloxy)tetradec-5-ynoate (1.13 g, 53% yield from bromide). TLC: 20% EtOAc/hexanes, R_{f}=0.63, charred with anisaldehyde. ¹H NMR (CDCl₃, 400 MHz) δ (0.88, t, J=6.8 Hz), 1.20-1.60 (m, 20H), 1.80 (quintet, 2H, J=7.1 Hz), 2.07-2.15 (m, 2H), 2.17-2.24 (m, 2H), 2.43 (t, 2H, J=7.2 Hz), 3.40 (t, 4H, J=6.8 Hz), 3.67 (s, 3H).

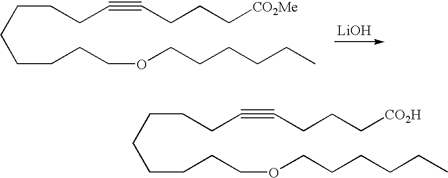

1N aq. LiOH (1.5 mL, 1.5 mmol) was added to a 0° C. solution of the above ester (110 mg, 0.32 mmol) in THF/H₂O (3:1, 12 mL) and then warmed to room temperature. After stirring overnight, the reaction mixture was acidified to pH 5 with 1N oxalic acid and extracted with ether. The combined ethereal extracts were washed with brine, dried over Na₂SO₄ and concentrated. The residue was purified by PTLC to give 14-(hexyloxy)tetradec-5-ynoic acid (14-HTYC) (100 mg, 95%) as a colorless oil. TLC: 40% EtOAc/hexanes, R_{f}=0.49, charred with anisaldehyde. ¹H NMR (CDCl₃, 400 MHz) δ 0.87 (t, 3H, J=7.2 Hz), 1.20-1.62 (m, 20H), 1.78 (quintet, 2H, J=7.1 Hz), 2.07-2.16 (m, 2H), 2.22-2.28 (m, 2H), 2.48 (t, 2H, J=7.2 Hz), 3.39 (t, 4H, J=6.8 Hz); ¹³C NMR (CDCl₃, 100 MHz) δ 14.26, 18.36, 18.89, 22.84, 24.16, 26.06, 26.34, 29.00, 29.23, 29.29, 29.58, 29.89, 29.90, 31.93, 32.94, 71.14, 71.18, 78.79, 81.68, 179.32; MS (CI) 325.2 (M+1).

### Example 7

### Additional Proposed Compounds of the Invention

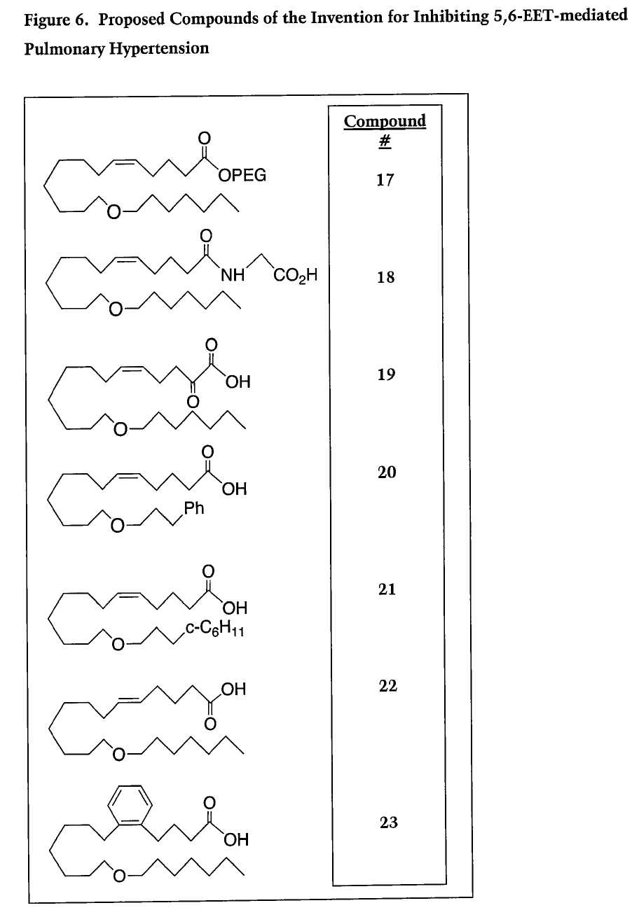

Additional proposed compounds useful for inhibiting 5,6-EET mediated pulmonary hypertension that are contemplated by this invention are shown in FIG. 6.

Compound 17 is an ester derivative of 13-HTEC (Compound 2) comprising a polyethylene glycol (PEG) ester of the C1 carboxylic acid of 13-HTEC. Compound 17 can be synthesized by modification of the synthesis outlined on pages 32-34 and methods described in Carey, F. A. and Sundberg, R. J. Advanced Organic Chemistry, Fourth Edition—Part B: Reaction and Synthesis, 2001. Polyethylene glycol ester derivatives of the free carboxylic acid group of 13-HTEC are represented herein by the formula —O—(CH₂—CH₂—O)ₙ—H, where n is between 1 and 15.

Compound 18 is an amide acid derivative of the C1 carboxylic acid of 13-HTEC. Compound 18 can be synthesized by modification of the synthesis outlined on pages 32-34 and methods described in Carey, F. A. and Sundberg, R. J. Advanced Organic Chemistry, Fourth Edition—Part B: Reaction and Synthesis, 2001. Similar methods can be used to synthesize other C2-C5 carboxy-amide derivatives of 13-HTEC.

Compound 19 is a C2 keto-derivative of 13-HTEC. Compound 19 can be synthesized by modification of the synthesis outlined on pages 32-34 and methods described in Carey, F. A. and Sundberg, R. J. Advanced Organic Chemistry, Fourth Edition—Part B: Reaction and Synthesis, 2001.

Compound 20 is a phenyl analog of 13-HTEC wherein C₁₇-C₂₀ are replaced with a phenol group. Compound 20 can be synthesized by modification of the synthesis outlined on pages 32-34 and methods described in Carey, F. A. and Sundberg, R. J. Advanced Organic Chemistry, Fourth Edition—Part B: Reaction and Synthesis, 2001.

Compound 21 is a cyclohexyl analog of 13-HTEC wherein C₁₇-C₂₀ are replaced with a cyclohexyl group. Compound 21 can be synthesized by modification of the synthesis outlined on pages 32-34 and methods described in Carey, F. A. and Sundberg, R. J. Advanced Organic Chemistry, Fourth Edition—Part B: Reaction and Synthesis, 2001.

Compound 22 is the trans-5,6-olefin analog of 13-HTEC. Compound 22 can be synthesized by modification of the synthesis outlined on pages 32-34 and methods described in Carey, F. A. Sundberg, R. J. Advanced Organic Chemistry, Fourth Edition—Part B: Reaction and Synthesis, 2001.

Compound 23 is an analog of 13-HTEC in which an ortho-substituted benzene replaces the 5,6-olefin. Compound 23 can be synthesized by modification of the synthesis outlined on pages 32-34 and methods described in Carey, F. A. and Sundberg, R. J. Advanced Organic Chemistry, Fourth Edition—Part B: Reaction and Synthesis, 2001.

### NON-PATENT PUBLICATIONS

Various patent and non-patent publications are cited herein, the disclosures of which are incorporated by reference in their entireties.
Barst, R. J. et al., Ann Intern Med 1994; 121:409-415
Bayat, M., and Cook, A. M. J., Neurosci. Nursing 36(4):231, 2004
Boschelli, D. H.; et al. Bioorganic & Medicinal Chemistry Letters (1992), 2(7), 705-8
Brashers, V. L., Peach, M. J., and Rose, C. E., Jr., Journal of Clinical Investigation, November 1988; 82(5): 1495-502
Carey, F. A. and Sundberg, R. J. Advanced Organic Chemistry, Fourth Edition—Part B: Reaction and Synthesis. 2001, Springer Publishing Company.
Corey, E. J. et al., J Am Chem Soc 101: 1586-1587, 1979
Dawson, C. A. et al., Journal of Applied Physiology 64: 274-284, 1988
Falck, J. R. et al., Am J Physiol. 2003; 284: H337-H349
Falck, J. R. et al. Synthesis of epoxyeicosatrienoic acids and heteroatom analogs. Methods Enzymol. 1990; 187:357-64.
Gauthier et al., Hypertension 42:555, 2003
Gessler T. et al., Eur Resp J 2001; 17:14-19
Herr, R. J., Bioorg Med Chem. 2002 November; 10(11):3379-93
Olschewski et al., Am. J. Respir. Crit. Care Med. 160(2): 600-607, 1999
Olschewski, H. et al., N Engl J Med. 2002 Aug. 1; 347(5):322-329
Pokreisz, P. et al., Hypertension 47:762-770, 2006
Stephenson, A. H. et al., American Journal of Physiology 275:H100, 1998
Stephenson, A. H. et al., American Journal of Physiology 284: H2153-H2161, 2003
Wermuth, C. G., “Molecular Variations Based on Isosteric Replacements” in The Practice of Medicinal Chemistry, 2^{nd} Edition, 2003, pp. 189-214
Wurtz, M. M. et al., J Appl Physiol 73: 2135-2141, 1992
Yaghi, et al., Journal of Pharmacology & Experimental Therapeutics 297: 479-488, 2001
Yaghi, A. et al., American Journal of Physiology—Lung Cell Mol Physiol 285: L1099, 2003
Zhu et al., American Journal of Physiology—Lung Cellular & Molecular Physiology 278: L335-L343, 2000

What is claimed is:

1. A method for decreasing pulmonary hypertension in a subject in need thereof, comprising the step of administering a composition comprising a pharmaceutically acceptable carrier and at least one compound selected from the group consisting of 13-heptyloxytridec-5(Z)-enoic acid (13-HTEC), 14-hexyloxyltetradec-5-ynoic acid (14-HTYC), pharmaceutically acceptable salts, esters, and acid derivatives thereof, thereby decreasing pulmonary hypertension in said subject.

2. The method of claim 1, wherein pulmonary hypertension is induced by hypoxia or by exposure of the subject to altitudes in excess of 1000 meters above sea level.

3. The method of claim 1, wherein the subject is a mammal selected from the group consisting of a rabbit, a sheep, a pig, a rat, a horse, a cow or a human.

4. The method of claim 1, wherein the composition is administered to the subject orally, topically, rectally, percutaneously, by parenteral injection, intranasally or by inhalation.

5. The method of claim 4, wherein the composition is administered by inhalation with a nebulizer, inhaler, dry powder inhaler, or metered dose inhaler.

6. The method of claim 1, wherein at least one test is used to identify subjects in need.

7. The method of claim 1, wherein at least one test is used to determine that administration of said composition results in a decrease in pulmonary hypertension in said subject.

8. The method of claim 7, wherein said test is selected from the group consisting of a six minute walk test, an echocardiogram, an electrocardiogram, a right heart catheterization, a pulmonary arterial pressure (PPA) test, a left atrial pressure (PLA) test, a central venous pressure (PCV) test, a systemic arterial pressure (PSYS) test, a heart rate (HR) test, a cardiac output (CO) test, a pulmonary artery wedge pressure (Ppa,we) test, a right ventricular ejection fraction (RVEF) test, and a central venous oxygen saturation (Sv_{O2}) test.

9. The method of claim 1, wherein the pharmaceutically acceptable esters of said compound are selected from the group consisting of a methyl ester, an ethyl ester, a propyl ester, an isopropyl ester, a butyl ester, an isobutyl ester, a t-butyl ester, a pentyl ester, a -cyclopropylethyl ester; a vinyl ester, an allyl ester, an ethynyl ester, a propynyl ester; a hydroxyethyl ester; a methoxymethyl ester, a 1-methoxyethyl ester, a phenyl ester, a tosyl ester, a t-butylphenyl ester, a salicyl ester, a 3,4-di-methoxyphenyl ester, a benzamidophenyl ester, a benzyl ester, a trityl ester, a benzhydryl ester, a —CHCHOHCH₂OH ester, a glycerol ester, and a polyethylene glycol ester.

10. The method of claim 1, wherein the pharmaceutically acceptable acid derivatives of said compound are selected from the group consisting of a —C₁-C₅ alkyl amide, a C₁-C₅ alkynyl amide, a C₁-C₅ alkenyl amide, an amino acid amide, hydroxamic acid (—NHOH), acyl-cyanamide (—NHCN), acylsulfonamide (—CO—NH—SO₂—R′), and methylsulfonimide.

11. A method of inhibiting 5,6-epoxyeicosatrienoic acid (EET)-mediated vasoconstriction of pulmonary arteries, comprising the step of administering a composition comprising an acceptable carrier and at least one compound selected from the group consisting of 13-heptyloxytridec-5(Z)-enoic acid (13-HTEC), thereby inhibiting 5,6-epoxyeicosatrienoic acid (EET)-mediated vasoconstriction of pulmonary arteries.

12. The method of claim 11, wherein the carrier is a pharmaceutically acceptable carrier and wherein epoxyeicosatrienoic acid (EET)-mediated vasoconstriction of pulmonary arteries is inhibited in a subject.

13. The method of claim 11, wherein the composition further comprises either PGF_{2α}, 5-hydroxytryptamine, or norepinephrine (NE) and wherein epoxyeicosatrienoic acid (EET)-mediated vasoconstriction of pulmonary arteries is selectively inhibited.

14. The method of claim 11, wherein epoxyeicosatrienoic acid (EET)-mediated vasoconstriction of pulmonary arteries is inhibited in an ex vivo system.

15. A compound 13-heptyloxytridec-5(Z)-enoic acid represented by the structure

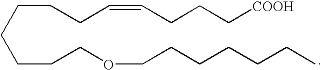

16. A composition comprising 13-heptyloxytridec-5(Z)-enoic acid or a pharmaceutically acceptable salt, ester, or acid derivative thereof, and a pharmaceutically acceptable carrier.

17. The composition of claim 16, wherein the pharmaceutically acceptable salt is selected from the group consisting of ammonium, sodium, potassium, calcium, magnesium, dicyclohexylamine, N-methyl-D-glucamine, arginine, lysine, methylamine, dimethylamine, cyclohexylamine, benzylamine, piperidine, ethylenediamine, ethanolamine, diethanolamine, triethanolamine, tris(hydroxymethylamino)ethane, monomethyl-monoethanolamine, procaine, caffeine and tetraalkyl ammonium salts, and combinations thereof.

18. The composition of claim 16, wherein the pharmaceutically acceptable ester is selected from the group consisting of methyl ester, ethyl ester, propyl ester, isopropyl ester, butyl ester, isobutyl ester, t-butyl ester, pentyl ester, -cyclopropylethyl ester; vinyl ester, allyl ester, ethynyl ester, propynyl ester; hydroxyethyl ester; methoxymethyl ester, 1-methoxyethyl ester, phenyl ester, tosyl ester, t-butylphenyl ester, salicyl ester, 3,4-di-methoxyphenyl ester, benzamidophenyl ester, benzyl ester, trityl ester, benzhydryl ester, —CHCHOHCH₂OH ester, glycerol ester, polyethylene glycol ester, and combinations thereof.

19. The composition of claim 16, wherein the pharmaceutically acceptable acid derivative is selected from the group consisting of —C₁-C₅ alkyl amide derivatives, C₁-C₅ alkynyl amide derivatives, C₁-C₅ alkenyl amide derivatives, amino acid amide derivatives, hydroxamic acid (—NHOH) derivative, acyl-cyanamide (—NHCN) derivative, acylsulfonamide (—CO—NH—SO₂—R′) derivative, methylsulfonimide derivative, and combinations thereof.

20. The composition of claim 16, wherein said pharmaceutically acceptable carrier comprises at least one solvent selected from the group consisting of ethanol, isopropanol, propylene glycol, polyethylene glycol, polypropylene glycol, glycol ether, and glycerol.

21. A compound represented by the structure: 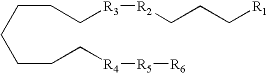
wherein R₁ is selected from the group consisting of —COOH, —C(O)O—(C₁-C₅ alkyl), —C(O)O—(C₁-C₅ alkenyls), —C(O)O—(C₁-C₅ alkynyl), —C(O)NHSO₂CH₃, —C(O)C₁-C₅ alkyl, —C(O)C₁-C₅ alkenyls, —C(O)C₁-C₅ alkynyl, —C(O)NHSO₂CH₃, —C(O)NHCH₂COOH, —C(O)NHCHCH₃COOH, —C(O)NHC(CH(CH₃)₂)COOH, —C(O)NHC(CH₂OH)COOH —NHC(CH(CH₃)OH)COOH, —C(O)OCHCHOHCH₂OH), a tetrazole, a 5-substituted-2,4-thiazolidinedione, an oxo-oxadiazole, an oxo-thiadiazole, a mercaptoazole, a sulfinylazole, a sulfonylazole, an isoxazole, an isothiazole, a hydroxy-thiadiazole, a hydroxy-chromone, a phosphinate, a phosphonate, a phosphonamide, a sulphonate, a sulphonamide, an acyl-sulphonamides and —C(O)O—(CH₂—CH₂—O)ₙ—H, where n is between 1 and 15; wherein —R₂-R₃— corresponds to —CH═CH— or —C≡C—; wherein —R₅— is —CH₂— or —O—; wherein —R₆ is an unbranched C₁-C₅ alkyl, an unbranched —O—C₁-C₅ alkyl, —CH₂CH₂CH₂C₆H₁₁, or —CH₂CH₂C₆H₁₀OH; wherein R₄ is —O— when R₅ is —CH₂— and R₆ is an unbranched C₁-C₅ alkyl, —CH₂CH₂CH₂C₆H₁₁, or —CH₂CH₂C₆H₁₀OH; wherein R₄ is —CH₂— when R₅ is —O— and R₆ is an unbranched C₁-C₅ alkyl; and wherein R₄ and R₅ are —CH₂— when R₆ is an unbranched —O—C₁-C₅ alkyl.

22. The compound of claim 21, wherein R₁ is —C(O)O—(CH2-CH2-O)ₙ—H, where n is between 1 and 15, wherein R₂-R₃— is —CH═CH—, wherein R₄ is —O—, wherein R₅ is —CH₂— and wherein R₆ is an unbranched C₆ alkyl.

23. The compound of claim 21, wherein R₁ is —C(O)NHCH₂COOH, wherein R₂-R₃— is —CH═CH—, wherein R₄ is —O—, wherein R₅ is —CH₂— and wherein R₆ is an unbranched C₆ alkyl.

24. The compound of claim 21, wherein R₁ is —COOH, wherein R₂-R₃— is —CH═CH—, wherein R₄ is —O—, wherein R₅ is —CH₂— and wherein R₆ is —CH₂CH₂CH₂C₆H₁₁.

25. The compound of claim 21, wherein R₁ is —COOH, wherein R₂-R₃— is —CH═CH—, wherein R₄ is —O—, wherein R₅ is —CH₂— and wherein R₆ is —CH₂CH₂C₆H₁₀OH.
